(19) United States
(12) Patent Application Publication
(10) Pub. No.: US 2016/0220154 A1
(43) Pub. Date: Aug. 4, 2016

(54) PORTABLE DEVICE, METHOD AND PROGRAM PRODUCT FOR GENERATING A PARAMETER RELATED TO THE PHYSICAL ACTIVITY OF A USER

(71) Applicant: Technogym USA Corp., Fairfield, NJ (US)

(72) Inventor: Alessandri Nerio, Cesena (FC) (IT)

(73) Assignee: Technogym USA Corp., Fairfield, NJ (US)

(21) Appl. No.: 14/610,450

(22) Filed: Jan. 30, 2015

(51) Int. Cl. A61B 5/11; A61B 5/00

(52) U.S. Cl. CPC A61B 5/1118; A61B 5/742; A61B 5/7278; A61B 2562/0219

(57) ABSTRACT

A portable device, method and program product for generating a parameter related to the physical activity of a user. The device includes at least one sensor configured to measure information representative of the movement of a user. The sensor is configured to generate, on the basis of such information, a plurality of values of physical activity data. A data processing unit of the device, operatively connected to the sensor, is configured to generate at least one rate activity parameter on the basis of the plurality of values of physical activity data generated by the sensor. The data processing unit is further configured to generate the rate activity parameter in a first reference time t0 on the basis of a portion of values of the plurality of values of physical activity data generated by the sensor in a set period of time preceding the first reference time t0.

### FIELD OF THE INVENTION

The present disclosure relates to the health and the physical activity of a user, particularly, to a portable device, method and program product for generating a parameter related to the physical activity of a user.

### BACKGROUND

Nowadays, many people are aware of the importance of healthy nutrition and sufficient physical activity.

In this regard, portable devices are known in the art which are able to monitoring the physical activity of a user, generating information representative of the performed physical activity.

Examples of such portable devices include a sensor (e.g. an accelerometer) configured to collect information representative of the movement of the user and a processing unit configured to generate and provide the user with information representative of the amount of the physical activity performed by the user.

However, nowadays, such portable devices and the information they are able to provide the user with are no longer sufficient and satisfactory.

Therefore, making available portable devices able to provide the user with more information about the performed physical activity in order to stimulate the physical activity of a user in a more precise and s reliable manner is strongly needed.

### SUMMARY OF THE INVENTION

According to some aspects of the present description, a portable device is provided which overcomes the above mentioned drawbacks of the prior art and in particular that is able to provide the user with more precise and reliable parameters relating to the physical activity performed by the user.

According to a first aspect, there is provided a portable device, comprising:
least one sensor for measuring information representative of the movement of a user, the at least one sensor being further configured to generate, on the basis of such information, a plurality of values of physical activity data,
a data processing unit, operatively connected to the at least one sensor, configured to generate at least one rate activity parameter on the basis of the plurality of values of physical activity data generated by the at least one sensor,
the data processing unit being further configured to generate the rate activity parameter in a first reference time t0 on the basis of a portion of values of the plurality of values of the physical activity data generated by the at least one sensor in a set period of time preceding the first reference time t0.

According to a second aspect, there is provided a method for generating a parameter related to the physical activity of a user, comprising steps of:
measuring, by the at least one sensor, information representative of the movement of a user;
generating, by the at least one sensor, on the basis of such information, a plurality of values of physical activity data;
generating, by the data processing unit, at least one rate activity parameter on the basis of the plurality of values of the physical activity data generated by the at least one sensor;
the step of generating comprises a step of generating, by the data processing unit, the at least a rate activity parameter in a first reference time t0 on the basis of a portion of values of the plurality of values of the physical activity data generated by the at least one sensor in a set period of time preceding the first reference time t0.

According to a third aspect, there is provided a program product loadable in a memory unit of an electronic calculator (e.g. a portable device such as a smartphone, a tablet, a PDA, a smartwatch, and so on), the program product being executed by a data processing unit of the electronic calculator to perform:
measuring, by the at least one sensor, information representative of the movement of a user;
generating, by the at least one sensor, on the basis of such information, a plurality of values of physical activity data;
generating, by the data processing unit, at least one rate activity parameter on the basis of the plurality of values of the physical activity data generated by the at least one sensor;
the step of generating comprises a step of generating, by the data processing unit, the at least a rate activity parameter in a first reference time t0 on the basis of a portion of values of the plurality of values of the physical activity data generated by the at least one sensor in a set period of time preceding the first reference time t0.

Further aspects are provided in the description, drawings and claims of the present application.

### BRIEF DESCRIPTION OF THE DRAWINGS

Further features and advantages of the portable device, the method and the program product according to the present disclosure will appear more clearly from the following description of preferred embodiments thereof, given by way of a non-limiting example with reference to the annexed figures, wherein:

FIG. 1 shows a schematic view of a portable device according to an embodiment of the disclosure;

FIG. 2 shows a block diagram representing the method for generating a parameter related to the physical activity of a user according to an embodiment of the disclosure.

### DETAILED DESCRIPTION OF THE PREFERRED EMBODIMENTS

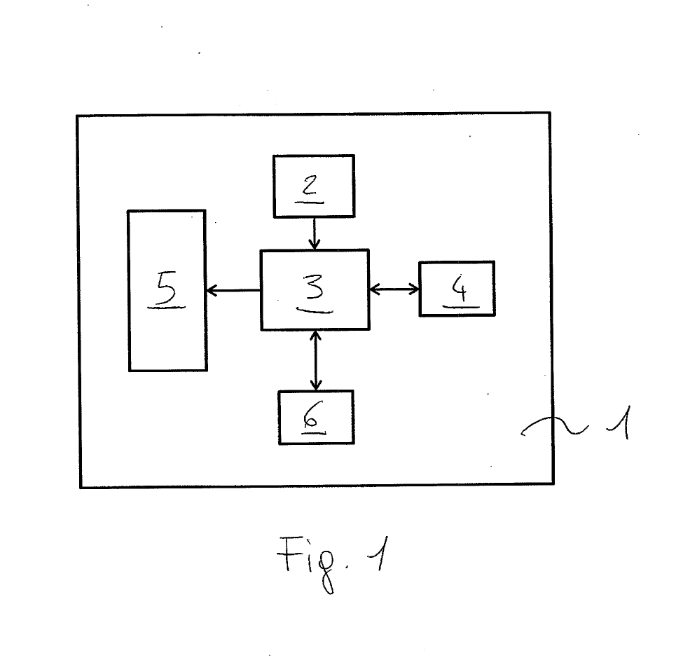

With reference to the embodiment of FIG. 1, a portable device 1 for generating a parameter related to the physical activity of a user, hereinafter also referred to simply as device 1, is now described.

It should be observed that in the embodiment of the FIG. 1, device 1 is represented as a block diagram only, in order to better highlight the technical features of the device which are important for the present disclosure.

The device of FIG. 1 can be a smartphone, a tablet, a smartwatch, a personal digital assistant (PDA), a multimedia digital player or any other electronic equipment which can be dressed by a user.

Device 1 comprises at least one sensor 2 for measuring information representative of the movement of a user.

Sensor 2 is further configured to generate, on the basis of such information, a plurality of values of physical activity data.

The physical activity data are generated on the basis of such information, related to the intensity of physical activity performed by the user.

It should be noted that such intensity of the physical activity may be expressed by the physiological measure expressing the energy cost of physical activities called MET (i.e. METabolic equivalent).

Sensor 2 can be an accelerometer (2D or 3D version), a gyroscope, a combination of these ones, or any other electronic element able to measure information representative of the movement of the user and to generate, on the basis of such information, a plurality of values of physical activity data.

Turning back to FIG. 1, device 1 further comprises a data processing unit 3, e.g. a microprocessor or a microcontroller, operatively connected to sensor 2, configured to generate at least one rate activity parameter on the basis of the plurality of values of the physical activity data generated by sensor 2.

In addition, data processing unit 3 is further configured to generate the rate activity parameter in a first reference time t0 on the basis of a portion of values of the plurality of values of the physical activity data generated by sensor 2 in a set period of time preceding the first reference time t0.

Such set period of time preceding the first reference time t0 may be stated as any period of time preceding the first reference time t0, e.g. 1 hour before the first reference time t0, half hour before the first reference time t0, 10 minutes before the first reference time t0, 1 minute before the first reference time t0, and so on.

This feature advantageously allows one to obtain a rate activity parameter representing the physical activity performed by the user in a set period of time preceding the instant time in which such parameter is generated (the first reference time t0), such as, in the last hour, in the last half hour, in the last minutes. i.e. providing at any time the user with an updated parameter related to the physical activity performed by the same user which can obtain a more precise and reliable information about that.

Turning again to the embodiment of FIG. 1, device 1 further comprises a memory unit 4, operatively associated to data processing unit 3. Memory unit 4 is configured to store both a program product (e.g. program code or a client software application) to allow device 1 to generate the parameter related to the physical activity of the user, as described below, and the data processed by data processing unit 3 during the operation of device 1.

In particular, memory unit 4 is configured to store the plurality of values of physical activity data generated by the sensor 2 and of course the rate activity parameter generated by data processing unit 3.

In addition, memory unit 4 is configured to store one or more client software applications to allow the user to enjoy digital services provided by the one or more client software application directly through device 1.

Furthermore, it is also confirmed that “client software application” means client software application of the standard type available on the market for electronic calculator and/or portable electronic device such as a personal computer, a notebook, a mobile phone (e.g. a smartphone), a tablet, a digital audio/video file reader (e.g. a MP3 reader), and so on.

Device 1 further comprises a display 5 configured to display to the user at least one of the following: the plurality of values of physical activity data generated by sensor 2 and the rate activity parameter generated by data processing unit 3.

Device 1 may comprises also a control panel, i.e. a keyboard or a graphical user interface, (not shown in the FIG. 1) configured to allow the user to interact with portable device 1. If the control panel has a touch screen, the control panel corresponds to display 5.

Device 1 may also comprises a data communication module 6 configured to communicate with other electronic devices and electronic calculator (e.g. a server computer), also arranged in a remote location, via a data communication channel in a wired or wireless mode.

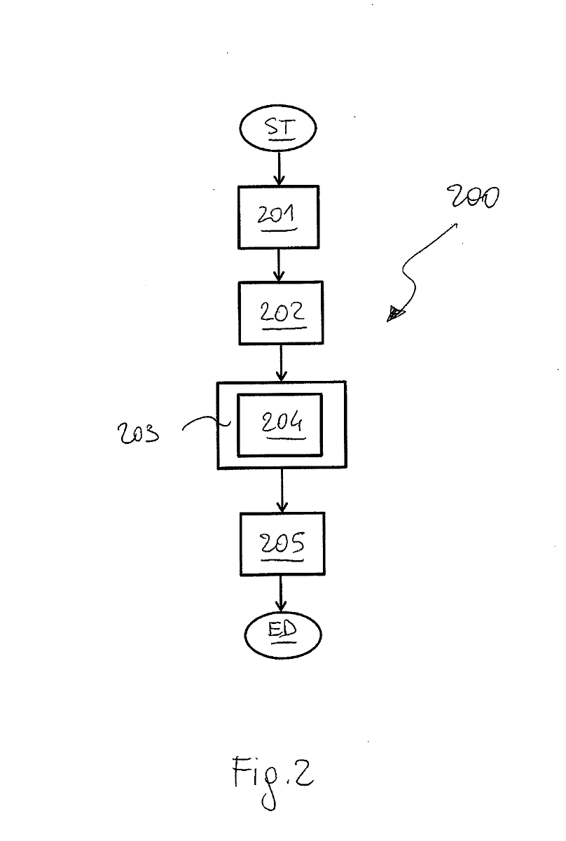

With reference now to FIG. 2, a method 200 for generating a parameter related to the physical activity of a user, in the following also simplified method 200, is described.

The method 200 comprises a symbolic step of starting ED.

The method 200 further comprises a step of measuring 201, by sensor 2, information representative of the movement of a user.

Furthermore, the method 200 comprises a step of generating 202, by sensor 2, on the basis of such information, a plurality of values of physical activity data.

The physical activity data are generated on the basis of such information, related to the intensity of physical activity performed by the user.

It should be noted that such intensity of the physical activity may be expressed by the physiological measure expressing the energy cost of physical activities called MET (i.e. METabolic equivalent).

Turning back to FIG. 2, the method 200 further comprises a step of generating 203, by data processing unit 3, at least one rate activity parameter on the basis of the plurality of values of the physical activity data generated by sensor 2.

Moreover, the step of generating 203 comprises a step of generating 204, by the data processing unit 3, the rate activity parameter in a first reference time t0 on the basis of a portion of values of the plurality of values of the physical activity data generated by sensor 2 in a set period of time preceding the first reference time t0.

The step of generating 203 further comprises a step of stating 205 such set period of time preceding the first reference time t0 as any period of time preceding the first reference time t0, e.g. 1 hour before the first reference time t0, half hour before the first reference time t0, 10 minutes before the first reference time t0, 1 minute before the first reference time t0, and so on.

The method 200 ends with a symbolic step of ending ED.

According to a further embodiment of the present disclosure, a program product can be loaded in the memory unit 4 of an electronic calculator (e.g. portable device 1, provided with data processing unit 2).

The program product can be executed by the data processing unit 2 of the electronic calculator (e.g. the data processing unit 2 of the portable equipment 1) to perform the steps of the method 200 for generating a parameter related to the physical activity of a user, previously described with reference to the embodiment of FIG. 2.

The portable device, the method and the program product according to the embodiments of the present disclosure allows the user to obtain a rate activity parameter representing the physical activity performed by the user in a set period of time preceding the instant time in which such parameter is generated (the first reference time t0), such as, in the last hour, in the last half hour, in the last minutes, i.e. providing at any time the user with an updated parameter related to the physical activity performed by the same user which can obtain a more precise and reliable information about the physical activity performed.

A man skilled in the art may make several changes, adjustments and replacements of elements with other functionality equivalent ones to the embodiments of the device, method and program product for managing data representative of the personal experience of a user on an exercise equipment described above in order to meet incidental needs, without departing from the scope of the following claims. Each of the features described as belonging to a possible embodiment can be obtained independently of the other embodiments described.

1. A portable device for generating a parameter related to the physical activity of a user, comprising:
at least one sensor for measuring information representative of the movement of a user, said at least one sensor being further configured to generate, on the basis of such information, a plurality of values of physical activity data,
a data processing unit, operatively connected to to said at least one sensor, configured to generate at least one rate activity parameter on the basis of said plurality of values of said physical activity data generated by said at least one sensor,
the data processing unit being further configured to generate said at least a rate activity parameter in a first reference time t0 on the basis of a portion of values of said plurality of values of said physical activity data generated by said at least one sensor 2 in a set period of time preceding the first reference time t0.

2. The device of claim 1, wherein said set period of time preceding the first reference time t0 may be stated as any period of time preceding the first reference time t0.

3. The device of claim 1, further comprising a memory unit, operatively associated to the data processing unit, said memory unit being configured to store both a program product to allow the device to generate the parameter related to the physical activity of the user and the data processed by the data processing unit during the operation of the device, said memory unit being further configured to store the plurality of values of physical activity data generated by the at least one sensor and said at least one rate activity parameter generated by the data processing unit.

4. The device of claim 1, further comprising a display configured to display to the user at least one of the following: the plurality of values of physical activity data generated by the at least one sensor and said at least one rate activity parameter generated by the data processing unit.

5. The device of claim 1, comprising also a control panel configured to allow the user to interact with the portable device.

6. The device of claim 1, further comprising a data communication module 6 configured to communicate with other electronic devices and electronic calculator via a data communication channel in a wired or wireless mode.

7. The device of claim 1, wherein the physical activity data are generated by said at least one sensor on the basis of such information, related to the intensity of physical activity performed by the user.

8. The device of claim 1, wherein said at least one sensor 2 comprises an accelerometer (2D or 3D version), a gyroscope, a combination of an accelerometer and gyroscope, or any other electronic element able to measure information representative of the movement of the user and to generate, on the basis of such information, a plurality of values of physical activity data.

9. The device of claim 1, wherein such device is a smartphone, a tablet, a smartwatch, a personal digital assistant (PDA), a multimedia digital player or any other electronic equipment which can be dressed by a user.

10. A method for generating a parameter related to the physical activity of a user, comprising steps of:
measuring, by said at least one sensor, information representative of the movement of a user;
generating, by said at least one sensor, on the basis of such information, a plurality of values of physical activity data;
generating, by the data processing unit, at least one rate activity parameter on the basis of said plurality of values of said physical activity data generated by said at least one sensor 2;
said step of generating comprises a step of generating, by the data processing unit, said at least a rate activity parameter in a first reference time t0 on the basis of a portion of values of said plurality of values of said physical activity data generated by said at least one sensor in a set period of time preceding the first reference time t0.

11. Program product loadable in the memory unit of an electronic calculator, the program product being executed by the data processing unit of the electronic calculator to perform:
measuring, by said at least one sensor, information representative of the movement of a user;
generating, by said at least one sensor, on the basis of such information, a plurality of values of physical activity data;
generating, by the data processing unit, at least one rate activity parameter on the basis of said plurality of values of said physical activity data generated by said at least one sensor 2;
said step of generating comprises a step of generating, by the data processing unit, said at least a rate activity parameter in a first reference time t0 on the basis of a portion of values of said plurality of values of said physical activity data generated by said at least one sensor in a set period of time preceding the first reference time t0.
